(19) United States
(12) Patent Application Publication
(10) Pub. No.: US 2009/0228048 A1
(43) Pub. Date: Sep. 10, 2009

(54) Joint Fixation System For the Hand

(76) Inventors: Scott F.M. Duncan, Owatonna, MN (US); Marianne V. Merritt, Scottsdale, AZ (US); Anthony A. Smith, Paradise Valley, AZ (US)

Correspondence Address: QUARLES & BRADY LLP, 411 E. WISCONSIN AVENUE, SUITE 2040, MILWAUKEE, WI, 53202-4497, US

(21) Appl. No.: 12/400,071

(22) Filed: Mar. 9, 2009

Related U.S. Application Data
(60) Provisional application No. 61/035,270, filed on Mar. 10, 2008.

(51) Int. Cl. A61B 17/58

(52) U.S. Cl. 606/286; 606/280

(57) ABSTRACT

A joint fixation system particularly useful for joints of the hand is disclosed. The joint fixation system includes a joint fixation plate having a distal section, a proximal section, and an intermediate section connecting the distal section and the proximal section. The distal section includes a distal section screw hole, and the proximal section includes a proximal section screw hole. The joint fixation plate is pre-bent such that the distal section and the proximal section form an included angle of less than 180 degrees. The joint fixation system includes a first screw dimensioned to engage an inner surface of the distal section screw hole, and a second screw dimensioned to engage an inner surface of the proximal section screw hole. The first screw is angulated proximally when inserted in the distal screw hole, and the second screw is angulated distally when inserted in the proximal screw hole.

### CROSS-REFERENCES TO RELATED APPLICATIONS

This application claims priority from U.S. Provisional Patent Application No. 61/035,270 filed Mar. 10, 2008.

### STATEMENT REGARDING FEDERALLY SPONSORED RESEARCH

Not Applicable.

### BACKGROUND OF THE INVENTION

1. Field of the Invention

The invention relates to devices for the surgical fixation of a joint to promote bone fusion, and more particularly to surgical devices for interphalangeal joint fusion and metacarpal phalangeal joint fusion.

2. Description of the Related Art

Arthrodesis, the surgical fixation of a joint to promote bone fusion, is often indicated for joint pain that cannot be managed by other treatments. Some causes of such joint pain are fractures and arthritis. Arthrodesis is commonly performed on joints in the hand, and ankle, and foot. With respect to the hand, arthrodesis may also be performed in the finger to correct digital abnormalities of the hand.

The hand includes a distal interphalangeal (DIP) joint, a proximal interphalangeal (PIP) joint, and a metacarpal phalangeal (MCP) joint in each finger, and an interphalangeal (IP) joint and a metacarpal phalangeal (MCP) joint in the thumb. The DIP joints, the IP joint, the PIP joints, and the MCP joints of the hand can be sites for osteoarthritis, and arthrodesis of the DIP, IP, PIP and MCP joints may be used to eliminate pain and improve the alignment and stability of the fingers and thumb. While joint arthroplasty for these joints of the hand may also be an option, there still are numerous cases that cannot be treated with a joint replacement. This is usually secondary to significant soft tissue loss or significant body destruction.

One current method for obtaining fusion in the interphalangeal and metacarpal phalangeal joints involves taking a small plate and having the surgeon bend it intraoperatively to approximate the angulation that is needed in the joint. The bony surfaces of the hand are prepared with a rongeur or saw, and then the plate is fixed across the joint by screws. Because this method results in a defined fused joint position, it is very important that the two adjacent bones are accurately positioned relative to each other during the method. However, it can be difficult to precisely control the angle when bending the plate, and often a “guesstimation” of the angle that is actually needed must be made by the surgeon.

Other techniques have the surgeon making bone cuts and then applying one screw in a compressive type fashion, again just with a rough estimation of the joint angle to be achieved. Finally, another common technique that is utilized is tension band wiring. Problems with tension band wiring include again, a “guesstimation” of the angle that is actually needed, as well as backing out the hardware, creating sore points or sore spots for the patient. These nonspecific techniques could result in nonunion of the fusion site. They can also lead to a mish-mash of different angles at different joints, which can make patient use of the hand less functional that would otherwise be achieved if the appropriate angles had been achieved.

Various joint fusion systems are available for hand joints. For example, an “MCP Fusion Plate”, designed for fusions of the first metacarpal phalangeal joint of the thumb is available from Acumed, Hillsboro Oreg., USA. U.S. Pat. No. 5,667,510 describes a joint fixation system having a screw assembly that provides a compressive force across a finger joint.

Various joint fusion systems are also available for other joints. A Hallu®-Fix arthrodesis set with an osteosynthesis plate indicated for use in the fixation of fractures, osteotomies and arthrodesis of the first metatarso-phalangeal (MTP) joint is available from Integra Life Sciences, Plainsboro, N.J., USA. MTP joint plates are also shown in U.S. Patent Application Publication Nos. 2003/0060827 and 2006/0241608. The use of MTP joint plates is also described by Kelikian in “Technical Considerations in Hallux Metatarsalphalangeal Arthrodesis”, Foot Ankle Clin N Am, Vol. 10, pages 167-190, 2005.

While these known joint fusion systems and methods may be acceptable for certain applications, there still exists a need for an improved device for the surgical fixation of an interphalangeal joint or a metacarpal phalangeal joint wherein the surgical devices provide greater precision in achieving a desired fused joint position.

### SUMMARY OF THE INVENTION

The present invention addresses the foregoing needs by providing a joint fusion system that supplies the hand surgeon, orthopedic surgeon and plastic surgeon with precontoured and angled joint fixation plates that provide easy ability to fuse interphalangeal or metacarpal phalangeal joints in the appropriate amount of angulation. The joint fixation system allows for easier and more reliable fusion of these joints in the appropriate individual angles for each digit.

In one aspect of the invention, there is provided a joint fixation system including a joint fixation plate having a distal section, a proximal section, and an intermediate section connecting the distal section and the proximal section. The distal section includes a distal section screw hole, and the proximal section includes a proximal section screw hole. The joint fixation plate is pre-bent such that the distal section and the proximal section form an included angle of less than 180 degrees. The distal section screw hole has a longitudinal axis that is angled with respect to a normal to a surface of the distal section, and the proximal section screw hole has a longitudinal axis that is angled with respect to a normal to a surface of the proximal section. In another form, only one of the distal section screw hole and the proximal section screw hole has a longitudinal axis that is angled with respect to a normal to a surface of the joint fixation plate. The joint fixation system includes a first screw dimensioned to engage an inner surface of the distal section screw hole, and a second screw dimensioned to engage an inner surface of the proximal section screw hole. The first screw is angulated proximally when inserted in the distal screw hole, and the second screw is angulated distally when inserted in the proximal screw hole such that the first screw and the second screw cross.

Because the joint fixation plate is pre-bent and has screw angles predetermined as well as an option for locking and unlocking screws, operating room time should be minimal compared to current techniques. Also, the accuracy of finger alignment and fusion rates should all be improved. The joint fixation plate has a very low profile in order to minimize the soft tissue irritation because of the very thin soft tissue envelope over the dorsum of the hand. For this same reason, the metal surfaces are rounded and polished. This allows the extensor tendons to glide over the plate without developing significant adhesions or showing signs of irritation.

In one aspect, the invention provides a joint fixation system for interphalangeal joint fusion or metacarpal phalangeal joint fusion. The system includes a joint fixation plate having a distal section, a proximal section, and an intermediate section connecting the distal section and the proximal section. The distal section engages one bone and includes a first distal section screw hole, and the proximal section engages another bone and includes a first proximal section screw hole. The distal section and the proximal section form an included angle less than 180 degrees. The first distal section screw hole has a longitudinal axis that is angled with respect to a normal to a surface of the distal section, and/or the first proximal section screw hole has a longitudinal axis that is angled with respect to a normal to a surface of the proximal section. Preferably, the longitudinal axis of the first distal section screw hole is angled with respect to the normal to the surface of the distal section, and the longitudinal axis of the first proximal section screw hole is angled with respect to the normal to the surface of the proximal section. In a lateral view of the joint fixation plate, the longitudinal axis of the first distal section screw hole and the longitudinal axis of the first proximal section screw hole cross within the included angle when the joint fixation plate is affixed across the joint.

In one configuration of the joint fixation system, the first distal section screw hole and the first proximal section screw hole are on opposite sides of a longitudinal axis of the joint fixation plate. The intermediate section of the joint fixation plate can be wider than the distal section, and the intermediate section of the joint fixation plate can be wider than the proximal section. The distal section can include a second distal section screw hole and a third distal section screw hole. The proximal section can include a second proximal section screw hole and a third proximal section screw hole.

In one form, the first distal section screw hole and the first proximal section screw hole are on opposite sides of a longitudinal axis of the joint fixation plate, and the first distal section screw hole and the second proximal section screw hole are on opposite sides of the longitudinal axis of the joint fixation plate, and the first proximal section screw hole and the second distal section screw hole are on opposite sides of a longitudinal axis of the joint fixation plate. The first distal section screw hole can be further from the longitudinal axis of the joint fixation plate than the second proximal section screw hole, and the first proximal section screw hole can be further from the longitudinal axis of the joint fixation plate than the second distal section screw hole.

The joint fixation system can further include a first screw dimensioned to engage an inner surface of the first distal section screw hole, and a second screw dimensioned to engage an inner surface of the first proximal section screw hole. In a lateral view of the joint fixation plate, when the first screw engages the inner surface of the first distal section screw hole and the second screw engages the inner surface of the first proximal section screw hole, the first screw and the second screw cross within the included angle of the joint fixation plate. The first screw can be a non-locking screw or a locking screw, and the second screw can be a non-locking screw or a locking screw.

The joint fixation system can further include a drill guide having threads for engaging threads on an inner surface of the first distal section screw hole and/or engaging threads on an inner surface of the first proximal section screw hole. The joint fixation system can further include another drill guide including a guide body having an end dimensioned for placement in the first distal section screw hole and/or the first proximal section screw hole. This drill guide also includes means for variably adjusting relationship of a longitudinal axis of the guide body to a longitudinal axis of the first distal section screw hole and/or to a longitudinal axis of the first proximal section screw hole.

In another aspect, the invention provides a kit for fixing one or more bone joints for fusion such as an interphalangeal joint or a metacarpal phalangeal joint. The kit includes two or more different joint fixation plates. A first joint fixation plate has a distal section, a proximal section, and an intermediate section connecting the distal section of the first joint fixation plate and the proximal section of the first joint fixation plate. The distal section of the first joint fixation plate can include a first distal section screw hole, and the proximal section of the first joint fixation plate can include a first proximal section screw hole. The distal section of the first joint fixation plate and the proximal section of the first joint fixation plate form a first included angle less than 180 degrees. The first distal section screw hole of the first joint fixation plate can have a longitudinal axis that is angled with respect to a normal to a surface of the distal section of the first joint fixation plate, and/or the first proximal section screw hole of the first joint fixation plate can have a longitudinal axis that is angled with respect to a normal to a surface of the proximal section of the first joint fixation plate. Preferably, the longitudinal axis of the first distal section screw hole is angled with respect to the normal to the surface of the distal section of the first joint fixation plate, and the longitudinal axis of the first proximal section screw hole is angled with respect to the normal to the surface of the proximal section of the first joint fixation plate.

A second joint fixation plate of the kit has a distal section, a proximal section, and an intermediate section connecting the distal section of the second joint fixation plate and the proximal section of the second joint fixation plate. The distal section of the second joint fixation plate can include a first distal section screw hole, and the proximal section of the second joint fixation plate can include a first proximal section screw hole. The distal section of the second joint fixation plate and the proximal section of the second joint fixation plate form a second included angle less than 180 degrees. The distal section screw hole of the second joint fixation plate has a longitudinal axis that is angled with respect to a normal to a surface of the distal section of the second joint fixation plate, and/or the proximal section screw hole of the second joint fixation plate has a longitudinal axis that is angled with respect to a normal to a surface of the proximal section of the second joint fixation plate. Preferably, the longitudinal axis of the first distal section screw hole is angled with respect to the normal to the surface of the distal section the second joint fixation plate, and the longitudinal axis of the first proximal section screw hole is angled with respect to the normal to the surface of the proximal section the second joint fixation plate.

In the kit, the first included angle of the first joint fixation plate and the second included angle of the second joint fixation plate kit are different. In addition, in the kit of the invention, any number of differently angled joint fixation plates can be included. For example, a differently angled joint fixation plate can be provided for each of the distal interphalangeal joint, the proximal interphalangeal joint, and the metacarpal phalangeal joint in each finger, and for the interphalangeal joint and the metacarpal phalangeal joint in the thumb. In one non-limiting example kit, joint fixation plates having included angles of 15°, 20°, 25°, 30°, 35°,40°, 45°, and 50° are provided.

The kit can include non-locking screws or locking screws for engaging the inner surface of the distal section screw hole of the first joint fixation plate and for engaging the inner surface of the proximal section screw hole of the first joint fixation plate and for engaging the inner surface of the distal section screw hole of the second joint fixation plate and for engaging the inner surface of the proximal section screw hole of the second joint fixation plate. The kit can include a drill guide having threads for engaging threads on an inner surface of the distal section screw hole of the first joint fixation plate and for engaging threads on an inner surface of the distal section screw hole of the second joint fixation plate.

In one form of the joint fixation plates in the kit, the longitudinal axis of the distal section screw hole of the first joint fixation plate is angled with respect to the normal to the surface of the distal section of the first joint fixation plate, and the longitudinal axis of the proximal section screw hole of the first joint fixation plate is angled with respect to the normal to the surface of the proximal section of the first joint fixation plate. The longitudinal axis of the distal section screw hole of the second joint fixation plate can be angled with respect to the normal to the surface of the distal section of the second joint fixation plate, and the longitudinal axis of the proximal section screw hole of the second joint fixation plate can be angled with respect to the normal to the surface of the proximal section of the second joint fixation plate.

In yet another aspect, the invention provides a method for fixing a bone joint for fusion. In the method, a joint fixation plate is affixed to adjacent bones of the bone joint, such as an interphalangeal joint or a metacarpal phalangeal joint. The joint plate has a distal section, a proximal section, and an intermediate section connecting the distal section and the proximal section. The distal section includes a distal section screw hole, and the proximal section including a proximal section screw hole. The distal section and the proximal section form an included angle of less than 180 degrees. The joint fixation plate can be provided pre-bent to the included angle. The distal section screw hole has a longitudinal axis that is angled with respect to a normal to a surface of the distal section, and/or the proximal section screw hole has a longitudinal axis that is angled with respect to a normal to a surface of the proximal section. In the method, a first screw is driven through the distal section screw hole such that the first screw engages an inner surface of the distal section screw hole and the adjacent bones, and a second screw is driven through the proximal section screw hole such that the second screw engages an inner surface of the proximal section screw hole and the adjacent bones. The distal section can include a second distal section screw hole, and the proximal section includes a second proximal section screw hole, and the method can further include the steps of driving a third screw through the second distal section screw hole such that the third screw engages an inner surface of the distal section screw hole and one of the adjacent bones, and driving a fourth screw through the second proximal section screw hole such that the fourth screw engages an inner surface of the second proximal section screw hole and one of the adjacent bones.

It is therefore an advantage of the invention to provide surgical methods and devices for interphalangeal and metacarpal phalangeal joint fusion wherein the devices provide greater precision in achieving a desired fused joint position.

It is another advantage of the invention to provide a joint fixation plate that can be used in cases where a prosthesis had to be removed because of infection or other reasons.

It is yet another advantage of the invention to provide a kit for interphalangeal and metacarpal phalangeal joint fusion wherein the kit includes joint fixation plates, screws, and a drill guide for drilling a pilot hole in each bone for receiving a screw.

These and other features, aspects, and advantages of the present invention will become better understood upon consideration of the following detailed description, drawings, and appended claims.

### BRIEF DESCRIPTION OF THE DRAWINGS

FIG. 1 is a lateral view of a left human index finger having a joint fixation plate according to the invention fixed at the proximal interphalangeal joint.

FIG. 2 is an antero-posterior view of the index finger of FIG. 1 having the joint fixation plate of FIG. 1 fixed at the proximal interphalangeal joint.

FIG. 3 is a cross-sectional view of the joint fixation plate taken along line 3-3 of FIG. 2.

FIG. 4 is a detailed view of the joint fixation plate taken along line 4-4 of FIG. 3.

FIG. 5 is a cross-sectional view, similar to FIG. 3, of the joint fixation plate before the mounting of a drill guide in an opening of the joint fixation plate.

FIG. 6 is a cross-sectional view, similar to FIG. 5, of the joint fixation plate after the mounting of a drill guide in a screw opening of the joint fixation plate.

FIG. 7 is a cross-sectional view, similar to FIG. 3, of the joint fixation plate before the mounting of another version of a drill guide in a screw opening of the joint fixation plate.

FIG. 8 is a cross-sectional view of the drill guide of FIG. 7 taken along line 8-8 of FIG. 7 with the drill guide body in the central position of the alignment base.

Like reference numerals will be used to refer to like parts from Figure to Figure in the following description of the drawings.

### DETAILED DESCRIPTION OF THE INVENTION

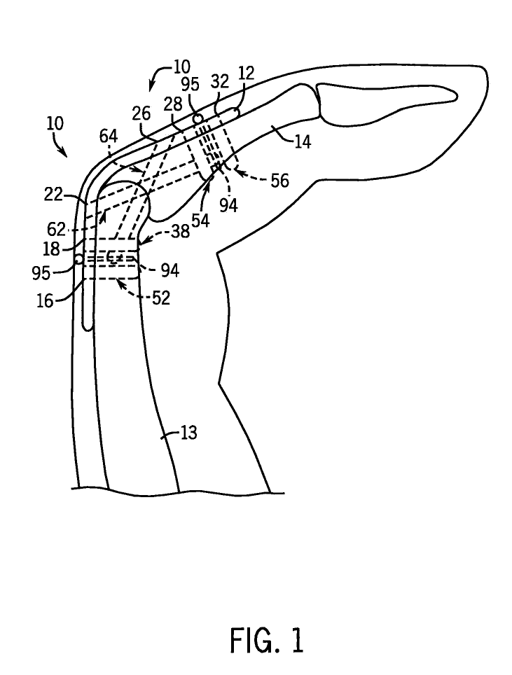

Looking first at FIGS. 1 to 4, there is shown one example embodiment of a joint fixation system 10 according to the invention. While the joint fixation system 10 is shown fixed on the proximal interphalangeal (PIP) joint of the left index finger, other embodiments of the invention can be used for any proximal interphalangeal (PIP) joint in the other fingers, a metacarpal phalangeal (MCP) joint in any finger, an interphalangeal (IP) joint in the thumb, or a metacarpal phalangeal (MCP) joint in the thumb.

The joint fixation system 10 includes a joint fixation plate 12. The surgical approach for the joint fixation system 10 can be dorsally for the IP, PIP and MCP joints. A cannulated cup and cone reamer system can be used to create matched mating surfaces for fusion of the opposed bone surfaces of the joint. Example reamer systems include: (1) the Small Joint Reamer available from Acumed, Hillsboro Oreg., USA; and (2) the Hallu®-Ream reamer system available from Integra Life Sciences, Plainsboro, N.J., USA. Different sized cannulated cup and cone reamer systems can provide appropriate surface preparation in different sized patients and different sized joints. The cannulated reamer system allows for consistent surface preparation. In the example of FIG. 1, the reamer system can be used create mating bone surfaces in the proximal phalanx 13 and the intermediate phalanx 14.

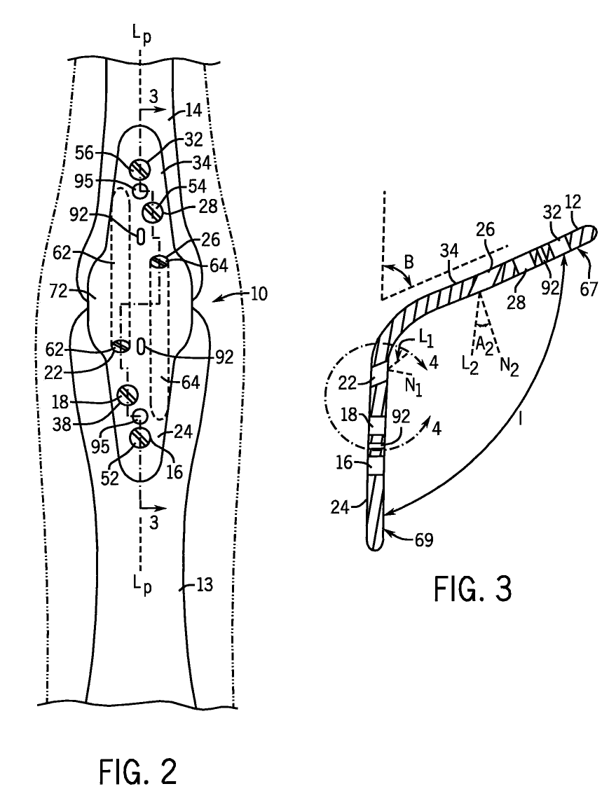

The joint fixation plate 12 of the joint fixation system 10 has a first screw hole 16, a second screw hole 18, and a third screw hole 22 on a proximal section 24 of the joint fixation plate 12. The joint fixation plate 12 also has a fourth screw hole 26, a fifth screw hole 28, and a sixth screw hole 32 on a distal section 34 of the joint fixation plate 12. The first screw hole 16, the second screw hole 18, the fifth screw hole 28, and the sixth screw hole 32 can be configured to accommodate either locking screws or non-locking screws. The joint fixation plate 12 of the joint fixation system 10 has a longitudinal axis Lₚ shown in FIG. 2. The proximal section 24 and the distal section 34 of the joint fixation plate 12 from an included angle I (see FIG. 3) of less than 180 degrees.

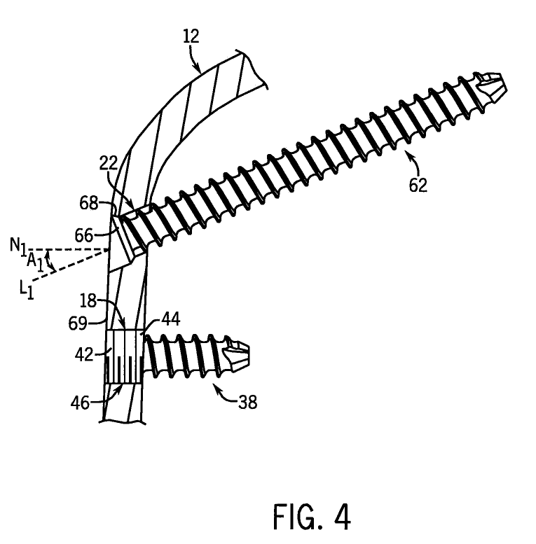

In FIG. 4, there is shown a locking cancellous second screw 38 threaded into the second screw hole 18 of the joint fixation plate 12. There are external threads 42 on the head 44 of the screw 38 which engage internal threads 46 on the interior surface of the second screw hole 18. This type of threaded engagement for the locking screw 38 and the second screw hole 18 can also be used for the first screw hole 16, the fifth screw hole 28, and the sixth screw hole 32. Looking at FIG. 1, it can be seen that the first screw hole 16, the fifth screw hole 28, and the sixth screw hole 32 engage the first locking cancellous screw 52, the fifth locking cancellous screw 54, and the sixth locking cancellous screw 56, respectively.

Advantageous aspects of the joint fixation system 10 include the third screw 62 and the fourth screw 64. The third screw 62 and the fourth screw 64 can be non-locking screws in order to provide compression. Preferably, the screw thread type is cancellous as shown in the third screw 62 in FIG. 4. The third screw 62 is angulated distally when inserted in the third screw hole 22 as shown in FIG. 4. The third screw hole 22 is countersunk to provide a chamfer 68 that engages frustoconical head 66 of the third screw 62. The fourth screw 64 is angulated proximally when inserted in the fourth screw hole 26 as shown in FIG. 1. The fourth screw hole 26 may also be countersunk to provide a chamfer that engages a frustoconical head of the fourth screw 64 (as in the third screw 62 and the third screw hole 22).

Looking at FIG. 2, the joint fixation plate 12 is widened laterally at an intermediate section 72 where the proximal section 24 and the distal section 34 of the joint fixation plate 12 are connected such that neither of the third screw 62 and the fourth screw 64 will interfere with the other when inserted into the proximal phalanx 13 and the intermediate phalanx 14.

The third screw hole 22 and the fourth screw hole 26 can be milled at specific angles A₁, A₂ (see FIGS. 3 and 4) depending on the angle of the joint fixation plate 12. For example, a plate angle B (see Angle B in FIG. 3) of 35° would require a much steeper angle of the compression screws in order to maintain the third screw 62 and the fourth screw 64 within the bony confines of the proximal phalanx 13 and the intermediate phalanx 14. Conversely, a greater fusion angle B, such as 50°, would require a less steep compression angle for the screws. Referring to FIG. 3, angle A₂ of the fourth screw hole 26 is created between a longitudinal axis L₂ of the fourth screw hole 26 and a normal N₂ to a surface 67 of the distal section 34. Referring to FIG. 4, angle A₁ of the third screw hole 22 is created between a longitudinal axis L₁ of the third screw hole 22 and a normal N₁ to a surface 69 of the proximal section 24. In an alternative embodiment (not shown), the longitudinal axis L₂ of the fourth screw hole 26 can be normal to the surface 67 of the distal section 34, and angle A₁ of the third screw hole 22 is created between the longitudinal axis L₁ of the third screw hole 22 and the normal N₁ to the surface 69 of the proximal section 24. In yet another alternative embodiment (not shown), the longitudinal axis L₁ of the third screw hole 22 can be normal to the surface 69 of the proximal section 24, and angle A₂ of the fourth screw hole 26 is created between the longitudinal axis L₂ of the fourth screw hole 26 and the normal N₂ to the surface 67 of the distal section 34.

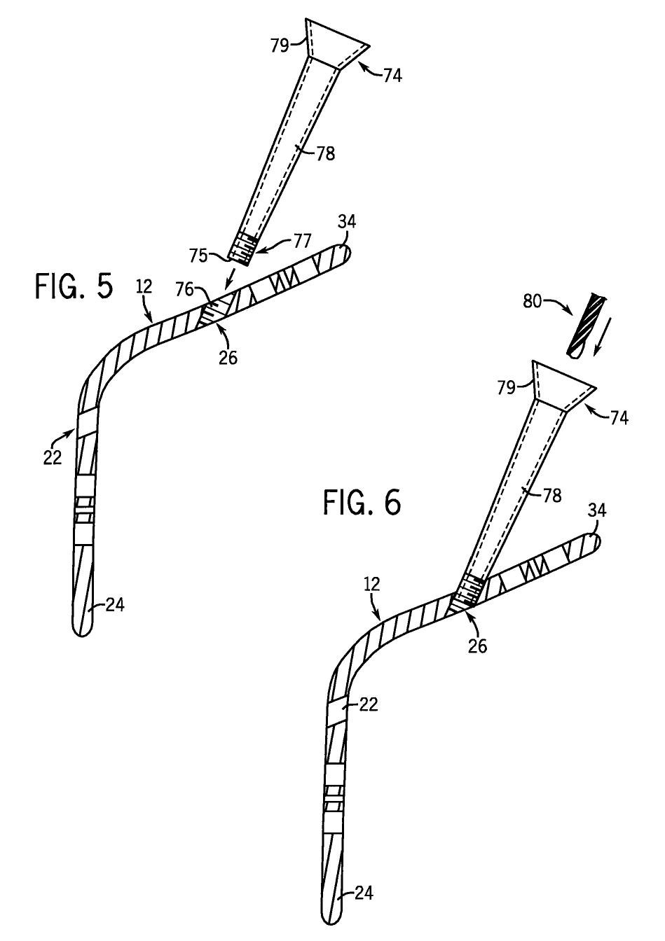

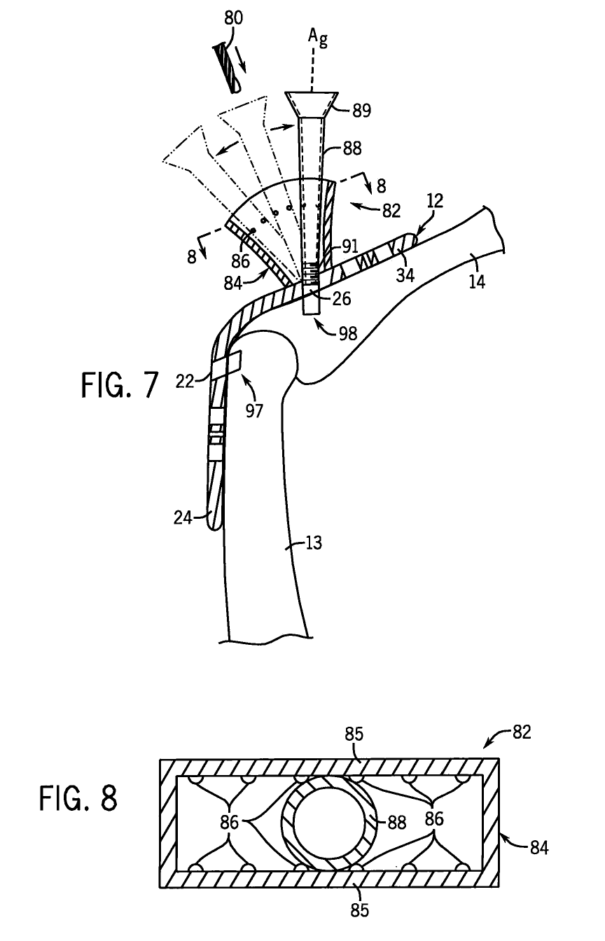

Looking at FIG. 5, the third screw hole 22 and the fourth screw hole 26 can have internal threads for screwing in a drill guide 74. In FIG. 5, the fourth screw hole 26 has internal threads 76 that engage external threads 77 on an end 75 of the drill guide 74. The drill guide 74 has a generally cylindrical tubular body 78 and a flared end 79. The body 78 of the drill guide 74 can taper slightly from the flared end 79 to the threaded end 75. As shown in FIG. 6, the drill guide 74 can be threaded into the fourth screw hole 26 to guide a drill 80 into bone to create a second screw guide bore 98 in the intermediate phalanx 14 (see FIG. 7). The end 75 of the drill guide 74 can be threaded into the third screw hole 22 to guide the drill 80 into bone to create a first screw guide bore 97 in the proximal phalanx 13 (see FIG. 7).

Another option is instead of designing the joint fixation plate 12 with a predetermined compression angle is the use of a variable angle drill guide 82. Looking at FIGS. 7 and 8, the drill guide 82 includes an alignment base 84 having spaced apart side walls 85. The interior surface of the side walls 85 includes a series of spaced protrusions 86. The drill guide 82 has a generally cylindrical tubular body 88 having a longitudinal axis A_{g}, and a flared end 89. The alignment base 84 is placed on the distal section 34 of the joint fixation plate 12 as shown in FIG. 7. An end 91 of the body 88 of the drill guide 82 is then placed in the fourth screw hole 26 and then the body 88 can be moved into an immobilized position between any adjacent spaced protrusions 86. Thus, the spaced protrusions 86 provide a means for variably adjusting the angular relationship of the longitudinal axis A_{g} of the guide body 88 to the longitudinal axis L₂ of the fourth screw hole 26. The body 88 can be positioned as desired to guide a drill 80 into the intermediate phalanx 14 to create a second screw guide bore 98 in the intermediate phalanx 14. Likewise, the variable angle drill guide 82 can be positioned with the body 88 in the third screw hole 22 at a desired angle to guide the drill 80 into the proximal phalanx 13 to create a first screw guide bore 97 in the proximal phalanx 13.

Whether using the drill guide 74 or the variable angle drill guide 82, a compression angle is created between the first screw guide bore 97 in the proximal phalanx 13 and the second screw guide bore 98 in the intermediate phalanx 14 so that the third screw 62 and the fourth screw 64 cross the joint at the midpoint of the joint so that the compressive forces applied across the joint surface area would be maximized. By maximizing these compression forces over the large prepared surface area, a higher success rate of obtaining fusion would be encountered clinically.

The joint fixation plate 12 can also have Kirschner wire (K wire) holes 92 (see FIGS. 2 and 3) that allow for temporization of the joint fixation plate 12 on the phalanx or metacarpals so that the third screw 62 and the fourth screw 64 could be placed prior to committing the first locking cortical or cancellous screw 52, the second locking cortical or cancellous screw 38, the fifth locking cortical or cancellous screw 54, and the sixth locking cortical or cancellous screw 56. These K-wire holes 92 can be oblong to create longitudinal play along axis Lₚ so that as the third screw 62 and the fourth screw 64 were are tightened, the K-wires would be able to accommodate this change of position. The K-wires 94 have globe shaped beads 95 (see FIG. 1) on their end so that the globe shaped beads 95 help hold the joint fixation plate 12 against the proximal phalanx 13 and the intermediate phalanx 14.

In order to attain functionality of the hand after fusion, different digits are fused at different angles. Non-limiting examples of the fusion angles for the proximal interphalangeal joint are: index finger at 35°, the long finger at 40°, the ring finger at 45°, and the small finger at 50°, with the thumb interphalangeal joint having a plate at 15°. Certainly, if the surgeon chooses to change the combination of plates, that would up to him or her. However, these plates should preferably not be bent by the surgeon because of the specific compression screw angles that would change if the angle of the plate were disturbed. Non-limiting examples of the fusion angles of the metacarpal phalangeal joints fusion are: 25° for the index finger, 30° for the long finger, 35° for the ring finger, and 40° for the small finger, with the thumb metacarpal phalangeal joint being fused at 15°. It is possible that one set of plates may be able to accommodate proximal interphalangeal and metacarpal phalangeal joint fusions. In this case, potentially having joint fixation plates angled from 15° to 50° in 5° increments, or at least from 20° to 50° in 5° increments would suffice for a majority of cases This would mean seven to eight different types of joint fixation plates being available. An example single kit can be supplied with: (1) any number (such as seven or eight) of different types of joint fixation plates 12; (2) locking screws (such as 38) of different lengths; (3) non-locking screws (such as 62) of different lengths; and (4) K-wires (such as 94) of different lengths.

The joint fixation plate and the screws may be formed from various materials such as, without limitation: (i) a metal or metal alloy such as a titanium alloy (e.g., titanium-6-aluminum-4-vanadium), a cobalt alloy, a stainless steel alloy, or tantalum; (ii) a nonresorbable ceramic such as aluminum oxide or zirconia; (iii) a nonresorbable polymeric material such as polyethylene or polyetheretherketone available under the product name PEEK-OPTIMA®; or (iv) a nonresorbable composite material such as a carbon fiber-reinforced polymers (e.g., polysulfone or PEEK-Optima®).

Thus, the invention provides a joint fusion system that supplies the hand surgeon, orthopedic surgeon and plastic surgeon with precontoured and angled joint fixation plates that provide easy ability to fuse interphalangeal or metacarpal phalangeal joints in the appropriate amount of angulation. The joint fixation system allows for easier and more reliable fusion of these joints in the appropriate individual angles for each digit.

Although the present invention has been described in detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. A joint fixation system comprising:
a joint fixation plate having a distal section, a proximal section, and an intermediate section connecting the distal section and the proximal section, the distal section including a distal section screw hole, the proximal section including a proximal section screw hole,
wherein the distal section and the proximal section form an included angle less than 180 degrees, and
wherein (i) the distal section screw hole has a longitudinal axis that is angled with respect to a normal to a surface of the distal section, and/or (ii) the proximal section screw hole has a longitudinal axis that is angled with respect to a normal to a surface of the proximal section.

2. The joint fixation system of claim 1 wherein:
the longitudinal axis of the distal section screw hole is angled with respect to the normal to the surface of the distal section.

3. The joint fixation system of claim 1 wherein:
the longitudinal axis of the proximal section screw hole is angled with respect to the normal to the surface of the proximal section.

4. The joint fixation system of claim 1 wherein:
the longitudinal axis of the distal section screw hole is angled with respect to the normal to the surface of the distal section, and
the longitudinal axis of the proximal section screw hole is angled with respect to the normal to the surface of the proximal section.

5. The joint fixation system of claim 4 wherein:
in a lateral view of the joint fixation plate, the longitudinal axis of the distal section screw hole and the longitudinal axis of the proximal section screw hole cross within the included angle.

6. The joint fixation system of claim 1 wherein:
the distal section screw hole and the proximal section screw hole are on opposite sides of a longitudinal axis of the joint fixation plate.

7. The joint fixation system of claim 1 wherein:
the intermediate section is wider than the distal section, and
the intermediate section is wider than the proximal section.

8. The joint fixation system of claim 1 wherein:
the distal section includes a second distal section screw hole, and
the proximal section includes a second proximal section screw hole.

9. The joint fixation system of claim 8 wherein:
the distal section includes a third distal section screw hole, and
the proximal section includes a third proximal section screw hole.

10. The joint fixation system of claim 8 wherein:
the distal section screw hole and the proximal section screw hole are on opposite sides of a longitudinal axis of the joint fixation plate, and
the distal section screw hole and the second proximal section screw hole are on opposite sides of the longitudinal axis of the joint fixation plate, and
the proximal section screw hole and the second distal section screw hole are on opposite sides of a longitudinal axis of the joint fixation plate.

11. The joint fixation system of claim 8 wherein:
the distal section screw hole and the proximal section screw hole are on opposite sides of a longitudinal axis of the joint fixation plate, and
the distal section screw hole is further from the longitudinal axis of the joint fixation plate than the second proximal section screw hole, and
the proximal section screw hole is further from the longitudinal axis of the joint fixation plate than the second distal section screw hole.

12. The joint fixation system of claim 1 further comprising:
a first screw dimensioned to engage an inner surface of the distal section screw hole, and
a second screw dimensioned to engage an inner surface of the proximal section screw hole.

13. The joint fixation system of claim 12 wherein:
in a lateral view of the joint fixation plate, when the first screw engages the inner surface of the distal section screw hole and the second screw engages the inner surface of the proximal section screw hole, the first screw and the second screw cross within the included angle.

14. The joint fixation system of claim 12 wherein:
the first screw is a non-locking screw or a locking screw, and
the second screw is a non-locking screw or a locking screw.

15. The joint fixation system of claim 1 further comprising:
a drill guide having threads for engaging threads on an inner surface of the distal section screw hole.

16. The joint fixation system of claim 1 further comprising:
a drill guide including a guide body having an end dimensioned for placement in the distal section screw hole, the drill guide also including means for variably adjusting relationship of a longitudinal axis of the guide body to a longitudinal axis of the distal section screw hole.

17. The joint fixation system of claim 1 wherein
the distal section includes a second distal section screw hole,
the proximal section includes a second proximal section screw hole, and
the system further comprises a first screw dimensioned to engage an inner surface of the second distal section screw hole, and
a second screw dimensioned to engage an inner surface of the second proximal section screw hole.

18. A kit for fixing one or more bone joints for fusion, the kit comprising:
a first joint fixation plate having a distal section, a proximal section, and an intermediate section connecting the distal section of the first joint fixation plate and the proximal section of the first joint fixation plate, the distal section of the first joint fixation plate including a distal section screw hole, the proximal section of the first joint fixation plate including a proximal section screw hole, wherein the distal section of the first joint fixation plate and the proximal section of the first joint fixation plate form a first included angle less than 180 degrees, wherein (i) the distal section screw hole of the first joint fixation plate has a longitudinal axis that is angled with respect to a normal to a surface of the distal section of the first joint fixation plate, and/or (ii) the proximal section screw hole of the first joint fixation plate has a longitudinal axis that is angled with respect to a normal to a surface of the proximal section of the first joint fixation plate; and
a second joint fixation plate having a distal section, a proximal section, and an intermediate section connecting the distal section of the second joint fixation plate and the proximal section of the second joint fixation plate, the distal section of the second joint fixation plate including a distal section screw hole, the proximal section of the second joint fixation plate including a proximal section screw hole, wherein the distal section of the second joint fixation plate and the proximal section of the second joint fixation plate form a second included angle less than 180 degrees, wherein (i) the distal section screw hole of the second joint fixation plate has a longitudinal axis that is angled with respect to a normal to a surface of the distal section of the second joint fixation plate, and/or (ii) the proximal section screw hole of the second joint fixation plate has a longitudinal axis that is angled with respect to a normal to a surface of the proximal section of the second joint fixation plate,
wherein the first included angle and the second included angle are different.

19. The kit of claim 18 further comprising:
screws for engaging the inner surface of the distal section screw hole of the first joint fixation plate and for engaging the inner surface of the proximal section screw hole of the first joint fixation plate and for engaging the inner surface of the distal section screw hole of the second joint fixation plate and for engaging the inner surface of the proximal section screw hole of the second joint fixation plate.

20. The kit of claim 18 further comprising:
a drill guide having threads for engaging threads on an inner surface of the distal section screw hole of the first joint fixation plate and for engaging threads on an inner surface of the distal section screw hole of the second joint fixation plate.

21. The kit of claim 18 wherein:
the longitudinal axis of the distal section screw hole of the first joint fixation plate is angled with respect to the normal to the surface of the distal section of the first joint fixation plate, and
the longitudinal axis of the proximal section screw hole of the first joint fixation plate is angled with respect to the normal to the surface of the proximal section of the first joint fixation plate.

22. The kit of claim 18 wherein:
the longitudinal axis of the distal section screw hole of the second joint fixation plate is angled with respect to the normal to the surface of the distal section of the second joint fixation plate, and
the longitudinal axis of the proximal section screw hole of the second joint fixation plate is angled with respect to the normal to the surface of the proximal section of the second joint fixation plate.

23. A method for fixing a bone joint for fusion, the method comprising:
affixing a joint fixation plate to adjacent bones of the bone joint,
wherein the joint plate has a distal section, a proximal section, and an intermediate section connecting the distal section and the proximal section, the distal section including a distal section screw hole, the proximal section including a proximal section screw hole,
wherein the distal section and the proximal section form an included angle less than 180 degrees,
wherein (i) the distal section screw hole has a longitudinal axis that is angled with respect to a normal to a surface of the distal section, and/or (ii) the proximal section screw hole has a longitudinal axis that is angled with respect to a normal to a surface of the proximal section.

24. The method of claim 23 further comprising:
driving a first screw through the distal section screw hole such that the first screw engages an inner surface of the distal section screw hole and the adjacent bones, and
driving a second screw through the proximal section screw hole such that the second screw engages an inner surface of the proximal section screw hole and the adjacent bones.

25. The method of claim 23 wherein:
the distal section includes a second distal section screw hole,
the proximal section includes a second proximal section screw hole, and
the method further comprises driving a third screw through the second distal section screw hole such that the third screw engages an inner surface of the distal section screw hole and one of the adjacent bones, and driving a fourth screw through the second proximal section screw hole such that the fourth screw engages an inner surface of the second proximal section screw hole and one of the adjacent bones.

26. The method of claim 23 wherein:
the bone joint is selected from an interphalangeal joint and a metacarpal phalangeal joint.

27. The method of claim 23 wherein:
the joint fixation plate is provided pre-bent to the included angle.
